(19) United States
(12) Patent Application Publication
(10) Pub. No.: US 2008/0187598 A1
(43) Pub. Date: Aug. 7, 2008

(54) Baby diaper cream for diaper itch and diaper red sore

(76) Inventor: Mary D. Hu, El Monte, CA (US)

Correspondence Address: DAVID AND RAYMOND PATENT FIRM, 108 N. YNEZ AVE., SUITE 128, MONTEREY PARK, CA, 91754, US

(21) Appl. No.: 11/701,734

(22) Filed: Feb. 1, 2007

(51) Int. Cl. A61K 36/355; A61K 35/56; A61K 36/00; A61K 36/232; A61K 36/634; A61K 36/288; A61K 36/328; A61K 36/33

(52) U.S. Cl. 424/547; 424/764; 424/767; 424/769; 424/773; 424/778

(57) ABSTRACT

A baby diaper cream for diaper itch and diaper red sore includes a predetermined amount of herbal medicine, comprising a predetermined amount of Forsythia and a predetermined amount of Honeysuckle, a predetermined amount of catalyst comprising vitamin E, and a predetermined amount of a binding agent.

### BACKGROUND OF THE PRESENT INVENTION

1. Field of Invention

The present invention relates to a baby diaper cream. More specifically, the present invention relates to a baby diaper cream for diaper itch and diaper red sore.

2. Description of Related Arts

Baby or hospital diuresis patients who are redridden for extended periods of time become afflicted with various skin maladies, such as wounds, sores, ulcers, and various surface conditions, as a result of poor blood and air circulation. The most common form of affliction is known as a diaper itch or diaper red sore. The part of the body making contact with a diaper or cloth is under pressure by the weight of the body urging against the diaper or cloth, thus resulting in muscle and associated tissue and becoming compressed and stiffened. Especially the diaper was moistened by urine, this situation is most acute when the bone of a patient presses on the tissue, such as the spine, coccyx or tailbone, hips, and stern. The weight of the patient's or baby's body presses upon the urinary diaper, which presses upon adjacent tissue and skin. As a result, blood circulation decreases and the tissue beings to decay. The patients and babies may also experience muscle pain and cramps as normal levels of oxygen and nutrition that would otherwise be delivered to muscles are decreased because of reduced blood circulation. The outer layer of the skin or epidermis becomes weakened and loses its resiliency and ability to sustain injury and heal.

Moreover, the part of the body making direct contact with the diaper or cloth is prevented from receiving an adequate supply or air or air circulation necessary to promote healing of any wounds, ulcers, or sores caused by an extended stay in a diaper.

The diaper itch and diaper red sore are generally characterized in severity over five stages: Stage 1. characterized by surface discoloration (pinkish or reddening) of the kin; Stage 2. broken or unbroken blistering; Stage 3, extension of wound through all layers of the skin; Stage 4. extension of wound through the skin, underlying muscle, tendon, and bone; and Stage 5. generally referring to an extremely deep wound affecting underlying organs and bone.

The most common method of prevention involves having the patient or baby change diaper and position over a regular interval, generally every two hours, so as to relieve pressure over the body and discard urinary diaper. In some cases, the patient or baby can change diaper unaided, but in many situations nursing care must be provided to help a patient change diaper at the prescribed interval. The changing of diaper typically involves rolling the patient, and easily has the baby cried, although other methods and procedures may be used depending upon the condition of the patient and baby.

However, not all patients or babies can change diaper at regular intervals to relieve pressure and discard urinary diaper so as to prevent the formation of an ulcer or to prevent the worsening of an existing ulcer. In such situations, a diaper cushion or other device may be positioned under or adjacent to the wound between the patient and the diaper red sore to relieve pressure or redistribute pressure away from the wound. Nevertheless, the diaper itch and diaper red sores may still develop even with use of such diaper cushions or devices, and topical treatment of the diaper itch and diaper red sores then becomes necessary.

A conventional method of treating the diaper itches and diaper red sores is to cover the itches and sores with a patch or dressings, while accomplishing their intended function, have their own drawbacks and disadvantages. For instance, patches and dressings completely cover a diaper itch and diaper red sore and thus prevent ambient air from reaching the diaper itch and diaper red sore, which would otherwise permit the diaper itch and diaper red sore the dry out and eventually heal. The diaper itch and diaper red sore thus remains as an open wound because of the lack of air, and healing is retarded indefinitely.

What is needed then to overcome the aforementioned disadvantages of patches and dressings for diaper itches and diaper red sores is the provision of a baby diaper cream that can be applied to a diaper itch and diaper red sore so as protect the diaper itch and diaper red sore from infection and to reduce inflammation and swelling, while allowing ambient air to reach the diaper itch and diaper red sore in order to promote eventual healing of the diaper itch and diaper red sore. However, the recently baby diaper cream for controlling and healing red sores, isn't aim at the diaper itch and diaper red sore. Therefore, it is necessary that find a useful baby diaper cream for curing diaper itch and diaper red sore.

### SUMMARY OF THE PRESENT INVENTION

The present invention provides a baby diaper cream, ointment, or salve having medicinal properties that promote healing of diaper itch and diaper red sore. The present invention overcomes all of the shortcomings described in the background of the invention, in addition to novel aspects that will be descried in detail hereinafter.

Baby diaper cream is a topical treatment that is to applied directly to a baby diaper itch and diaper red sore, or the like in order to promote healing thereof. The baby diaper cream is comprised of at least two different primary Chinese herbs containing anti-biotic and anti-inflammatory agents so as to prevent infection and swelling, a catalyst that activates the medicinal and healing capabilities of the herbs and a carrier and binding agent that holds the herbs and catalyst together and against the diaper red sore and adjacent tissue. Furthermore, the carrier and binding agent allow eventual passage of air therethrough so that said air can reach the diaper red sore located under the cream so as to promote drying and healing of the diaper red sore. The baby diaper cream can be enhanced by addition of one or more secondary Chinese herbs to the primary Chinese herbs.

The baby diaper cream is applied directly over and to the diaper red sore, ulcer, or the like. The anti-biotic and anti-inflammatory ingredients of the herbs bone function reduce swelling and prevent infection of the diaper red sore. The carrier or binding agent of the cream permits passage of ambient air therethrough so that air eventually makes contact with the diaper red sore under the cream and tissue adjacent to the cream so as to promote drying and eventual healing of the diaper red sore.

Furthermore, the aforementioned embodiments of the baby diaper cream can be modified by adding one or more secondary Chinese herbs, adjusting either the number of herbs and particular quantities thereof to the four embodiments of the diaper red sore cream described previously. These additional herbs include common Chinese herbs, such as Da Qing Ye, Ban Lan Gen, Qing Dai, Pu Gong Ying, Zi Hua Di Ding, Yu Xing Cao, Bai Jiang Cao, Ma Chi Xian, Tu Fu Ling, Bai Xian Pi, She Gan, San Dou Gen, Chuan Xin Lian, Dan Gui, Hu Zhang, Jiang Xiang, Bai Tou Weng, and the like.

Accordingly, it is an object of the invention to provide a low-cost, easy-to-manufacture, and easy-to-market baby diaper cream.

A further object of the invention is to provide an easy-to-use, versatile, and adaptable baby diaper cream.

Another object of the invention is to provide a baby diaper cream in various embodiments comprised of a mixture of two or more primary Chinese Herbs, a catalyst and a binding or carrier agent, said primary herbs comprised in equal or varying parts of Lian Qiao, Yi Yi Ren, Mo Yao, Hai Piao Xiao, Jin Yin Hua, and Bai Zhi, and a variety of secondary Chinese herbs.

Another object of the invention is to provide a baby diaper cream that can be applied to diaper red sore, wound, ulcer, and the like so as to prevent infection and to reduce inflammation and swelling, while allowing ambient air to reach the diaper red sore in order to dry the diaper red sore and promote eventual healing thereof.

Accordingly, in order to accomplish the above objects, the present invention provides a baby diaper cream directly applicable on a skin condition prone area of a user, wherein the baby diaper cream treats skin conditions on the skin condition prone area, wherein the baby diaper cream comprises:

a predetermined amount of herbal medicine which comprises a predetermined amount of Forsythia and a predetermined amount of Honeysuckle, wherein the Forsythia has a Chinese herb name of Lian Qiao which has forsythin, matairesinoside, betulinic acid, phyillygenin, and pinoresinol, wherein the Forsythia is provided for lowering fever, promoting anti-inflammation, protecting the liver, ceasing vomiting and promoting blood circulation and urination, and the Honeysuckle has a Chinese herb name of Jin Yin Hua, provided for treating abscesses, sores and inflammation;

a predetermined amount of catalyst comprising vitamin E; and

a predetermined amount of a binding agent.

### DETAILED DESCRIPTION OF THE PREFERRED EMBODIMENT

The herbs function as the primary herbs within the invention and can be dried and processed into a powdery or granular texture and combined with two or more of each other granular texture and combined with two or more of each other in specific quantities to achieve a particular medicinal, therapeutic effect. A binding agent and carrier are added to the combination of two or more hers to facilitate application and efficacy of the primary herbs. The baby diaper cream created with the primary herbs can be enhanced with the addition of one or more secondary Chinese herbs in varying quantities, as will be referenced later in this description.

The baby diaper cream according to the present invention is provided in the following embodiments or compounds, including in equal (or varying) parts or quantities of said primary herbs and a catalyst and binding or carrier agent, such as a cream or gel, which functions to contain the mixture of herbs and catalyst and bind to the diaper red sore and surrounding tissue layer, thereby ensuring that the mixture of herbs and catalyst make contact with the afflicted area and provide a healing effect. The carrier or binding agent also allows ambient air to reach the diaper red sore so as to promote drying and eventual healing of the diaper red sore.

According to the present embodiment, the baby diaper cream includes equal or varying parts of Forsythia having a Chinese herb name of Lian Qiao and Honeysuckle having a Chinese herb name of Jin Yin Hua, a predetermined amount of catalyst comprising vitamin E, and a predetermined amount of binding agent, such as gel and cream, mixing with the powder form Forsythia, the powder Honeysuckle and the catalyst to form the baby diaper cream.

The Lian Qiao and the Jin Yin Hua are dried and processed to a fine or granular powder. Lian Qiao, commonly known in Western taxonomy as Forsythia, provides a broad spectrum antibiotic effect and can be used to lower fever, promote anti-inflammation, protect the liver, stop vomiting, and promote blood circulation and urination. Its chemical ingredients include forsythin, matairesinoside, betulinic acid, phyillygenin, and pinoresinol. Jin Hin Hua is commonly known in Western taxonomy as Honeysuckle, which flowers, leaves, and bark are used. The herb is used primarily to treat “hot” infectious disorders, such as abscesses, sores, and inflammation.

In one embodiment, the baby diaper cream comprises 40% to 50% by weight of the binding agent, 5% to 10% by weight of the catalyst and 40% to 50% by weight of the herbal medicine.

According to the present invention, the herbal medicine may further include a predetermined amount of powder form Myrrh, having a Chinese herb name of Mo Yao, which is commonly known in Western taxonomy as Myrrh, functions as an anti-inflammatory and anti-bacterial agent, reducing pain and swelling and also potent enough to soften swellings and carbuncles, wherein the herbal medicine includes 33% of the Forsythia, 33% of the Honeysuckle and 33% of the Myrrh.

According to the present invention, the herbal medicine may further include a predetermined amount of powder form Garden Angelica having a Chinese herb name of Bai Zhi, which is commonly known in Western taxonomy as Garden Angelica, Wild Angelica, and Root of the Holy Ghost, alleviates pain, clears and reduces swelling and inflammation, expels pus from carbuncles, sores, and ulcers, and expels dampness. It can be used topically as a multi-ingredient preparation. The part of the plant providing the medicinal and therapeutic effect is the root. In which, the herbal medicine includes 33% of the Forsythia, 33% of the Honeysuckle and 33% of the Garden Angelica. Alternatively, the herbal medicine may include 25% of the Forsythia, 25% of the Honeysuckle, 25% of the Garden Angelica, and 25% of the Myrrh.

According to the present invention, the herbal medicine may further comprise a predetermined amount of powder form Cuttlefish Bone having a Chinese herb name of Hai Piao Xiao or Wu Zei Gu, which is commonly known in Western taxonomy as Cuttlefish bone, is known to have properties that stop bleeding, restore kidney deficiencies, and promote healing of rashes and ulcers. In which, the herbal medicine includes 25% of the Forsythia, 25% of the Honeysuckle, 25% of the Garden Angelica, and 25% of the Cuttlefish Bone. Alternatively, the herbal medicine may include 20% of the Forsythia, 20% of the Honeysuckle, 20% of the Garden Angelica, 20% of the Cuttlefish Bone, and 20% of the Myrrh.

According to the present invention, the herb medicine may further comprise additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.

In one embodiment, the baby diaper cream for diaper itch and diaper red sore, including a predetermined amount of herbal medicine which includes a predetermined amount of powder form Garden Angelica having a Chinese herb name of Bai Zhi and a predetermined amount of powder form Cuttlefish Bone having a Chinese herb name of Hai Piao Xiao, a predetermined amount of catalyst comprising vitamin E, and a predetermined amount of a binding agent mixing the Garden Angelica and the Cuttlefish Bone to form the baby diaper cream.

According to the present invention, the baby diaper cream comprises 40% to 50% by weight of the binding agent, 5% to 10% by weight of the catalyst and 40% to 50% by weight of the herbal medicine, wherein the binding agent can be gel or cream such as Vaseline.

According to the present invention, the herbal medicine further comprises a predetermined amount of Jobstears Seed having a Chinese herb name of Yi Yi Ren. Yi Yi Ren, Jobstears Seed is the dried ripe kernel obtained by removing the hard husk of the fruit and seed coat of Coix lacryma-jobi L. var. ma-yuen (Roman.) Stapt (Gramineae). The herb provides for lowering fever, promoting anti-inflammation, moistening, and evacuating purulence and carbuncles. In which, the baby diaper cream comprises 40% to 50% by weight of the binding agent, 5% to 10% by weight of the catalyst and 40% to 50% by weight of the herbal medicine which comprises 33% of the Garden Angelica, 33% of the Cuttlefish Bone and 33% of the Jobstears Seed.

According to the present invention, the herb medicine may further include additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knotweed, and dalbergia heartwood.

A quantity of the baby diaper cream in the aforementioned embodiment is applied directly over and to the diaper red sore, wound, or the like. The medicinal and therapeutic properties of the cream will promote healing of the diaper red sore and obviate the need for closed dressings or patches. The baby diaper cream can be re-applied as needed to ensure that healing of the diaper red sore continues unabated. If necessary, the quantity of any particular herb in relation to quantities of other herbs within a particular embodiment or compound can be varied to customize or tailor the medicinal affects of a particular embodiment of the baby diaper cream. For instance, increasing or decreasing the quantity of a particular herb within a particular embodiment of the baby diaper cream will likewise increase or decrease its medicinal and therapeutic function within said embodiment of the baby diaper cream.

Similarly, the aforementioned embodiments of the baby diaper cream can be enhanced by adding one or more of the following secondary herbs, adjusting either the number of herbs and particular quantities thereof to the four embodiments or the diaper red sore cream described previously. These additional herbs include, but are not limited to the following:

Da Qing Ye (Isatis leaf)

Ban Lan Gen (Isatis root)

Qing Dai (Natural Indigo)

Pu Gong Ying (Dandelion)

Zi Hua Di Ding (Viola)

Yu Xing Cao (Houttuynia)

Bai Jiang Cao (Thiaspi)

Ma Chi Xian (Portulaca)

Tu Fu Ling (Smilax)

Bai Xian Pi (Dictamnus)

She Gan (Belamcanda, Blackberry Lily)

San Dou Gen (Sophora Root)

Chuang Xin Lian (Green Chiretta)

Dang Gui (Angelica Root)

Hu Zhang (Japanese Knottweed)

Jiang Xiang (Dalbergia Heartwood)

Bai Tou Weng (Chinese Anemone Root)

While the present invention has been illustrated and described in detail in the foregoing description, the same is to be considered as illustrative and not restrictive in character, it being understood that only the preferred embodiments have been shown and described and that all changes and modifications that come within the spirit of the invention are desired to be protected. With respect to the above description then, it is to be realized that the optimum relationships for the ingredients of the invention, to include variations in quantity, texture, function and use, are deemed readily apparent and obvious to one skilled in the art, and all equivalent relationships to those illustrated in the drawings and described in the specification are intended to be encompassed by the present invention.

From the foregoing, it will be understood by persons skilled in the art that an improved baby diaper cream has been provided. The invention is relatively simple and easy to create, yet affords a variety of uses. While my description contains many specificities, these should not be construed as limitations on the scope of the version of the invention, but rather as an exemplification of the preferred embodiment thereof. The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be resorted to, falling within the scope of the invention. Although this invention has been described in its preferred form with a certain degree of particularity, it is understood that the present disclosure of the preferred forms have been made only by way of example and numerous changes in the details of construction and combination and arrangement of components and ingredients may be resorted to without departing from the spirit and scope of the invention.

What is claimed is:

1. A baby diaper cream for diaper itch and diaper red sore, including:
a predetermined amount of herbal medicine which comprises a predetermined amount of powder form Forsythia having a Chinese herb name of Lian Qiao and a predetermined amount of powder form Honeysuckle having a Chinese herb name of Jin Yin Hua;
a predetermined amount of catalyst comprising vitamin E; and
a predetermined amount of binding agent mixing with said powder form Forsythia, said powder Honeysuckle and said catalyst to form said baby diaper cream.

2. The baby diaper cream, as recited in claim 1, wherein said baby diaper cream comprises 40% to 50% by weight of said binding agent, 5% to 10% by weight of said catalyst and 40% to 50% by weight of said herbal medicine.

3. The baby diaper cream, as recited in claim 2, wherein said binding agent is a gel.

4. The baby diaper cream, as recited in claim 2, wherein said binding agent is a cream.

5. The baby diaper cream, as recited in claim 1, wherein said herbal medicine further includes a predetermined amount of powder form Myrrh, having a Chinese herb name of Mo Yao.

6. The baby diaper cream, as recited in claim 5, wherein said baby diaper cream comprises 40% to 50% in weight of said binding agent, 5% to 10% in weight of said catalyst and 40% to 50% in weight of said herbal medicine which includes 33% of said Forsythia, 33% of said Honeysuckle and 33% of said Myrrh.

7. The baby diaper cream, as recited in claim 1, wherein said herbal medicine further includes a predetermined amount of powder form Garden Angelica having a Chinese herb name of Bai Zhi.

8. The baby diaper cream, as recited in claim 7, wherein said baby diaper cream comprises 40% to 50% in weight of said binding agent, 5% to 10% in weight of said catalyst and 40% to 50% in weight of said herbal medicine which includes 33% of said Forsythia, 33% of said Honeysuckle and 33% of said Garden Angelica.

9. The baby diaper cream, as recited in claim 5, wherein said herbal medicine further comprises a predetermined amount of powder form Garden Angelica, having a Chinese herb name of Bai Zhi.

10. The baby diaper cream, as recited in claim 9, wherein said baby diaper cream comprises 40% to 50% in weight of said binding agent, 5% to 10% in weight of said catalyst and 40% to 50% in weight of said herbal medicine which includes 25% of said Forsythia, 25% of said Honeysuckle, 25% of said Garden Angelica, and 25% of said Myrrh.

11. The baby diaper cream, as recited in claim 1, wherein said herbal medicine further comprises a predetermined amount of powder form Cuttlefish Bone having a Chinese herb name of Hai Piao Xiao.

12. The baby diaper cream, as recited in claim 7, wherein said herbal medicine further comprises a predetermined amount of powder form Cuttlefish Bone having a Chinese herb name of Hai Piao Xiao.

13. The baby diaper cream, as recited in claim 12, wherein said baby diaper cream comprises 40% to 50% in weight of said binding agent, 5% to 10% in weight of said catalyst and 40% to 50% in weight of said herbal medicine which includes 25% of said Forsythia, 25% of said Honeysuckle, 25% of said Garden Angelica, and 25% of said Cuttlefish Bone.

14. The baby diaper cream, as recited in claim 9, wherein said herbal medicine further comprises a predetermined amount of powder form Cuttlefish Bone having a Chinese herb name of Hai Piao Xiao.

15. The baby diaper cream, as recited in claim 14, wherein said baby diaper cream comprises 40% to 50% in weight of said binding agent, 5% to 10% in weight of said catalyst and 40% to 50% in weight of said herbal medicine which includes 20% of said Forsythia, 20% of said Honeysuckle, 20% of said Garden Angelica, 20% of said Cuttlefish Bone, and 20% of said Myrrh.

16. The baby diaper cream, as recited in claim 1, further comprising additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.

17. The baby diaper cream, as recited in claim 14, further comprising additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.

18. A baby diaper cream for diaper itch and diaper red sore, including:
a predetermined amount of herbal medicine which includes a predetermined amount of powder form Garden Angelica having a Chinese herb name of Bai Zhi and a predetermined amount of powder form Cuttlefish Bone having a Chinese herb name of Hai Piao Xiao;
a predetermined amount of catalyst comprising vitamin E; and
a predetermined amount of a binding agent mixing said Garden Angelica and said Cuttlefish Bone to form said baby diaper cream.

19. The baby diaper cream, as recited in claim 18, wherein said baby diaper cream comprises 40% to 50% by weight of said binding agent, 5% to 10% by weight of said catalyst and 40% to 50% by weight of said herbal medicine.

20. The baby diaper cream, as recited in claim 18, wherein said herbal medicine further comprises a predetermined amount of Jobstears Seed having a Chinese herb name of Yi Yi Ren.

21. The baby diaper cream, as recited in claim 20, wherein said baby diaper cream comprises 40% to 50% by weight of said binding agent, 5% to 10% by weight of said catalyst and 40% to 50% by weight of said herbal medicine which comprises 33% of said Garden Angelica, 33% of said Cuttlefish Bone and 33% of said Jobstears Seed.

22. The baby diaper cream, as recited in claim 18, further comprising additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knotweed, and dalbergia heartwood.

23. The baby diaper cream, as recited in claim 20, further comprising additional herbs selected from a group consisting of isatis leaf, isatis root, natural indigo, dandelion, viola, houttuynia, thlaspi, partulaca, smilax, dictamnus, belamcanda, sophora root, green chiretta, angelica root, Japaneses knotweed, and dalbergia heartwood.
